(19) United States
(12) Patent Application Publication
(10) Pub. No.: US 2026/0034326 A1
(43) Pub. Date: Feb. 5, 2026

(54) INHALATION MASK

(71) Applicant: PARI Gmbh SPEZIALISTEN FÜR EFFEKTIVE INHALATION, Starnberg (DE)

(72) Inventors: Christian Peter, Alling (DE); Andrea Winzen, Gräfelfing (DE)

(73) Assignee: PARI Gmbh, Starnberg (DE)

(21) Appl. No.: 19/100,071

(22) PCT Filed: Aug. 1, 2023
(86) PCT No.: PCT/EP2023/071258

§ 371 Date: Jan. 30, 2025

(30) Foreign Application Priority Data
Aug. 3, 2022 (EP) 22188533.8

(51) Int. Cl. A61M 16/06; A61M 15/00

(52) U.S. Cl. CPC A61M 16/0616; A61M 16/0611; A61M 15/0086; A61M 2205/0227; A61M 2205/584

(57) ABSTRACT

Inhalation mask (1) for an inhalation system (14) comprising a mask body (2) with a port (3) that can be connected to the inhalation system (14), and an opening (4) with a scaling lip (5) being equipped for joining a face (9) in such a way that the nose, the mouth or both of them are surrounded by the sealing lip (5), the scaling lip (5) comprising a stowing area (6) and a remaining area (7) arranged adjacent to the stowing area (6), wherein in a sealing position the stowing area (6) shows one or more of the conditions laying on the face, being compressed or bent to an inner side of the inhalation mask (1) and the inhalation mask (1) is provided with an identification (8) distinguishing the stowing area (6) from the remaining area (7).

The present invention concerns an inhalation mask for an inhalation system, an inhalation assembly and a usage of an inhalation mask.

Inhalation masks are commonly used for connecting inhalation systems like chambers, spacers, MDIs or inhalers such as jet or mesh inhalers to faces of users. The inhalation mask should provide a connection from the inhalation system to the user's respiration system through which an aerosol can flow. Leakage between the inhalation mask and the face should be minimized.

Medical guidelines recommend the usage of inhalation masks especially for children's inhalation. Particularly people with difficulties in coordinating their breathing benefit from using an inhalation mask. With many inhalation masks, a user can inhale an aerosol from an inhalation system through the mouth as well as through the nose.

The interface to the user's face is often designed of a flexible material formed as a lip. The combination of this easily deformable structure with a soft material allows a flexible adaption of the inhalation mask to the face geometry und thus a high tightness.

Currently many inhalation masks are used in an inefficient way. They are pressed to hard or soft onto faces resulting in an insufficient sealing.

Due to the flexibility and the adapted material characteristics, very high pressing forces can compress the lip so much that with a multicomponent mask a hard component can come into direct contact with the face of a patient reducing wearing comfort and tightness. Reduced tightness can occur with insufficient pressing forces as well.

The present invention is directed towards providing an alternative inhalation mask to improve the situation described above.

This object is achieved by an inhalation mask for an inhalation system comprising a mask body with a port that can be connected to the inhalation system, and an opening with a sealing lip being equipped for joining a face in such a way that the nose, the mouth or both of them are surrounded by the sealing lip, the sealing lip comprising a stowing area and a remaining area arranged adjacent to the stowing area, wherein in a sealing position the stowing area shows at least in one section one or more of the conditions laying on the face, being compressed or bent to an inner side of the inhalation mask and the inhalation mask is provided with an identification distinguishing the stowing area from the remaining area.

By pressing the inhalation mask onto the face, particularly onto the face of a child, the lip is deformed and may be compressed. Therefore, the visible part of the lip is reduced. The distance being required for the deformation of the lip correlates directly with the force being applied and with the tightness. This is due to the geometry of the lip and the material characteristics. The identification shows if an adequate pressure is applied. Therefore, this inhalation mask provides a tool for the user or a caregiver that helps to realize an efficient connection between a face and the mask.

Visualization of a good mask position helps preventing, that caregivers, especially parents press the inhalation mask onto the face, especially a child's face with too high forces, as this leads to a reduced wearing comfort as well as leakage. Thus, adherence and therapy success can be improved. Besides caregivers can get a sense of security regarding correct positioning of the mask.

Improved wearing comfort can lead to a higher adherence. Caregivers can find out the intense or the force needed to press the mask onto the face for achieving a tight position having a high wearing comfort for the user or child and at the same time satisfies the necessary tightness for a high effectiveness of the medical therapy.

Inhalation masks are particularly used in the treatment of respiratory conditions or diseases affecting humans as adults, children, toddlers, infants or newborns. They can be used for the treatments of animals as well.

An inhalation mask includes a mask body shaped to receive a user's nose, mouth or both of them. An exterior surface of the mask body is exposed to an ambient environment.

Inhalation masks are available in different specifications: For example, with and without valves, holes and other openings. The interface to the user's face is often designed of a flexible material formed as a lip. They are used for different inhalation systems as jet nebulizers, membrane nebulizers, spacers, chambers, and others.

Inhalation masks differ in their materials and structure. Common structures are one material structures or multicomponent structures. Polyvinyl chloride (PVC), silicone, polycarbonate (PC), polypropylene (PP), thermoplastic elastomer (TPE) and other materials are used in inhalation masks.

The inhalation system preferably comprises an aerosol generation device. An aerosol generating device preferably comprises a nebulizer, a sprayer, a humidifier, a compressed air nebulizer, an air atomizer, an electronic nebulizer, an ultrasonic nebulizer, an electrohydrodynamic nebulizer, an electrostatic nebulizer, a membrane nebulizer, a nebulizer with a vibrating membrane, an electronic nebulizer with a vibrating membrane, a mesh nebulizer, a jet nebulizer, an MDI, an inhaler, a powder atomizer, a dry powder inhaler (DPI) or a combination thereof. The MDI comprises a pressurized canister with a medicament and a propellant.

The inhalation system may comprise a device for providing aerosols. The device for providing aerosols preferably comprises a spacer or a chamber. Devices for providing aerosols are preferably devices being arranged for use with MDI's. Devices for providing aerosols comprise spacers or chambers, arranged to receive aerosol preferably supplied by MDI's, so that users can inhale the aerosol.

Spacers do not have any valves. Chambers or holding chambers have inspiratory valves and preferably also expiratory valves.

Aerosols for therapeutic purposes are generated and delivered to a desired location within a user or patient's body with aerosol delivery devices. Aerosols are mixtures of solid or liquid particulates and a gas. Aerosols are preferably intended for application onto or into parts of the human or animal body, such as skin, body cavities, body openings, nose, paranasal sinuses, maxillary sinuses, frontal sinuses, sphenoidal sinuses, ethmoid sinuses, pharynx, larynx, trachea, lung, main bronchus, bronchi, bronchioles, alveoli, joints or abdominal cavity.

Aerosols may be employed, to prevent, diagnose or treat diseases of humans and animals, or to immunize against diseases. They are useful for the treatment of respiratory conditions or diseases affecting adults, children, toddlers, infants or newborns.

Preferably, the sealing lip comprises a soft component like an elastic material that returns to its original size and shape when an influence or force is removed. The elastic material may comprise silicone or an elastomer, especially a thermoplastic elastomer.

The soft component may comprise a transparent material and a nontransparent material. One of these materials may be arranged in the stowing area and the other material may be arranged in the remaining area.

The sealing lip comprises a stowing area, being deformed when the mask is pressed on a face correctly. The stowing area is equipped for being deformed such that it lays on the face, is bent to an inside of the mask body or is compressed when being pressed on a face correctly. Some or all of these may occur together. This deformation effects that the stowing area of the sealing lip becomes invisible or is at least harder to view from an outside of the mask. The mask may be formed at least partly by a transparent material. In one embodiment the stowing area is formed by a transparent material. When the mask is deformed in a correct way, the transparent material is not viewable. When laying on the face or being bent to an inside of the mask body, the stowing area may still be viewable through this material but is at least harder to view.

The remaining area is a part of the sealing lip that is arranged adjacent to the stowing area and has contact to the outside environment of the mask when it is used correctly.

The identification allows distinguishing the stowing area from the remaining area. This can be realized in multiple ways. Preferably, the stowing area and the remaining area are optically designed in a different way. In one embodiment, they are designed to feel differently. In one embodiment, a separation of stowing area and remaining area can be seen or felt. The separation may be a rib, notch, groove or line.

The two areas may show different roughnesses, or a glossy finish may be applied to one of the areas. Also, a coloring may be provided in one of the areas. The coloring can be provided only in some areas of the identification, so that for example stripes or dots appear in the identification area. Also, it is possible to provide different surface structures or textures. The stowing area or the remaining area may be transparent.

Further embodiments of the identification comprise one more of the following identification elements: grooves, recesses, bulges, bubbles and embossments. The recesses, bulges, bubbles and embossments may be circular or oval. The identification elements may be provided in the soft component. They visualize the required deformation of the mask. Identification elements in different dimensions, which are arranged by size and distance to a boundary between a remaining area and a stowing area can provide the user with a better information regarding actual status and the status of a good pressure for sealing. The identification may comprise rows of circular bulges. The bulges may be arranged parallel to a boundary between a remaining area and a stowing area. The bulges that are in the closest row to the boundary may have the biggest diameter. The bulges in the row being furthest away from the boundary may show the smallest diameter and the bulges in the row being between these two rows exhibit a diameter with dimensions that are between the dimensions of the bulges in the closest row and the furthest row. This way the user sees the different dimensions of the bulges that are being stowed away and thus disappearing with growing pressure. Therefore, it may be easier for the user to assess how much more pressure he should apply when he is mounting the mask.

Certainly, it is possible to combine several options.

In one embodiment the identification comprises two different materials.

In one embodiment the identification comprises a material out of the following materials silicone and thermoplastic elastomer for the stowing area and another material out of polyvinyl chloride, polycarbonate and polypropylene for the remaining area. In one embodiment two materials with different surface feels are used for the identification.

According to an embodiment the identification may comprise a first color for the stowing area and a different color for the remaining area.

The identification may comprise a marking or a surface structure.

The marking or surface structure may be provided in the stowing area or the remaining area. In one embodiment, the marking or surface structure is provided in the stowing area and the remaining area. It may be appropriate to provide different configurations of the marking or surface structure in different areas. In one embodiment, the marking or surface structure is arranged as a pattern. The marking or surface structure may comprise different material thicknesses, different surface roughness's, a matt area, a shiny area, a polished area, a bubble, a bulge, a protuberance, a recess, a color, a finish, an imprint or a scratch.

In an embodiment the marking or surface structure comprises several elements.

The several elements or items may be dots, polygons, ellipses, circles, bulges or recesses.

The marking or surface structure may at least to some extend be provided in the remaining area, wherein the dimensions of the elements are related to their distance to the stowing area.

The dimensions of elements arranged closer to the stowing area may be bigger than the dimensions of elements arranged further away from the stowing area. This can give an indication regarding the pressing force left to a good sealing.

The surface structure may comprise a rib, notch or groove.

The rib may be arranged between the stowing area and the remaining area. In one embodiment several ribs are provided on the remaining area.

In an embodiment the identification is invisible from the outside of the mask body when the mask is pressed on the face in a correct way.

The identification may be a color or marking arranged in the stowing area. When the mask is pressed on the face in a correct way, the stowing area with the identification should be invisible or at least harder to view, as it lays on the face, is compressed or bent to the inside of the mask body.

One aspect of the invention is an inhalation assembly comprising an inhalation mask and an inhalation system. The inhalation system may be a system being arranged for providing a medicament, a gas or an aerosol. Preferably the inhalation system comprises a metered-dose inhaler, a chamber, a spacer, a nebulizer, an atomizer, a jet nebulizer or a vibrating-mesh nebulizer.

It is an aspect of the invention to provide a method for using an inhalation mask comprising the steps of pressing the inhalation mask on a face in such a way that the nose, the mouth or both of them are surrounded by the sealing lip and adjusting the pressure by watching the identification such that the stowing area is deformed that it is invisible from an outside of the mask or loses contact to an ambient environment outside the mask and the remaining area is still viewable or in contact with the ambient environment.

By watching the identification, a caregiver can increase the pressure when he sees that the stowing area is still visible. On the other hand, he can reduce the pressure when he sees that the remaining area is not visible in full. When the remaining area is in contact with the ambient environment, it is possible to see it.

In one embodiment, the identification comprises a deformation indicator or tightness indicator preferably located on a flexible lip marking the correct position visualizing the deformation of the lip for caregivers or parents. The inhalation mask may be equipped with a soft, deformable component for visualization of the deformation path. The required position is marked on the lip.

The tightness indicator can support to visualize the required deformation path for achieving a high tightness by marks on the lip. This can be achieved by a circumferential marking on the lip, disappearing by compressing the lip, so that caregivers or parents can apply the mask with the correct force.

Embodiments of the identification comprise color marks, symbols, naps, recesses, grooves or lines. Recesses or naps may be realized as bubbles. Furthermore, it is suggested to use geometrically forms, which can be pushed together for constituting a defined graphic or contour in the correct position. Surface textures as for example eroded structures can be used as identification as well.

In one embodiment the identification comprises a marking or surface structure, which shows a symbol when the inhalation mask is pressed on the face correctly. This can be realized by structures or markings, which are distanced from each other when the seal is unstressed. When stress is applied to the seal, the structures or markings can move closer together, so that a symbol is created.

In a preferred embodiment the sealing lip rolls in towards the inner side of the mask when pressure is applied, and the mask contacts a face. When the sealing lip rolls in, the stowing area rolls in first. When the mask is pressed further towards the face, the remaining area starts rolling in as well. Stowing area and remaining area may roll towards the inner side of the mask when the pressure is applied accordingly. If the pressure is applied correctly, ideally the stowing area is rolled in completely and the remaining area is not rolled in at all.

In an embodiment the grade of rolling in correlates to the pressure used to press the mask onto the face. The sealing lip may roll in more with higher pressure und may roll in less with less pressure. For rolling in of the stowing area less pressure may be required than for rolling in of the remaining area. The pressure may increase continuously with the movement in direction to the face. In one embodiment the remaining area is stiffer than the stowing area. In this embodiment the pressure increases abruptly when the remaining area starts rolling in.

Preferably the sealing lip is made of one material, there is no difference in the materials of stowing area and remaining area. Parts of the mask or the entire mask may be fluorinated. Preferably, there is no coating.

In one embodiment there is no additional material in form of ripples or corrugations in the sealing lip for allowing a distortion to fit closely to a face especially around the nose.

### BRIEF DESCRIPTION OF THE DRAWINGS

FIG. 1 shows an inhalation mask with an identification;

FIG. 1a shows a view of an identification being formed like waves;

FIG. 1b shows a view of an identification being formed like bubbles;

FIG. 1c shows a view of an identification being colored;

FIG. 1d shows a view of an identification being formed as a line, rib, groove or notch;

FIG. 2 shows an inhalation mask with an identification being arranged in a distance from the sealing border;

FIG. 2a shows a view of an identification being formed like waves;

FIG. 2b shows a view of an identification being formed like bubbles;

FIG. 2c shows a view of an identification being colored;

FIG. 2d shows a view of an identification being formed as several lines or small ribs;

FIG. 3 shows an inhalation mask with an identification being arranged on the remaining area;

FIG. 3a shows a view of an identification being formed like waves;

FIG. 3b shows a view of an identification being formed like bubbles;

FIG. 3c shows a view of an identification being colored;

FIG. 4 shows an inhalation mask with an identification;

FIG. 4a shows a view of an identification being formed like waves;

FIG. 4b shows a view of an identification being formed like bubbles;

FIG. 4c shows a view of an identification being colored;

FIG. 4d shows a view of an identification being formed as a line;

FIG. 5 shows an inhalation mask with an identification;

FIG. 5a shows a view of an identification being formed like waves;

FIG. 5b shows a view of an identification being formed like bubbles;

FIG. 5c shows a view of an identification being colored;

FIG. 6 shows a sideview of an inhalation mask;

FIG. 7 shows a view of an inhalation mask being applied to a face;

FIG. 8 shows a sideview of an inhalation mask with a sealing lip being adapted to the contour of a face;

FIG. 9 shows the inhalation mask of FIG. 8 being applied to a face;

FIG. 10 shows a sideview of an inhalation mask with a sealing lip being adapted to the contour of a face;

FIG. 11 shows the inhalation mask of FIG. 10 being applied to a face;

FIG. 12 shows an inhalation assembly with a chamber;

FIG. 13 shows an inhalation assembly; and

FIG. 14 shows an inhalation assembly with a nebulizer.

### DETAILED DESCRIPTION OF THE PREFERRED EMBODIMENTS OF THE INVENTION

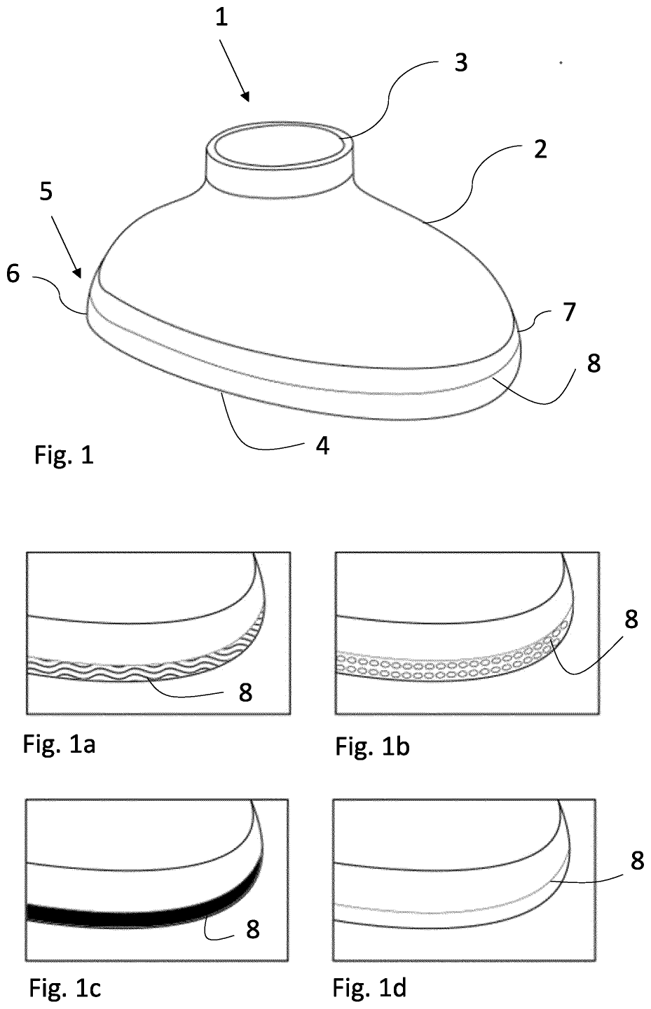

FIG. 1 shows an inhalation mask 1 with a mask body 2, a port 3 that can be connected to an inhalation system, an opening 4 and a circumferential sealing lip 5 being arranged at the opening 4. The circumferential sealing lip 5 is intended for applying the mask to the face of a user. It shows a stowing area 6 and a remaining area 7.

When the mask 1 is pressed onto a face with an adequate force, the stowing area 6 cannot be seen by the user anymore. It lays on the face or is compressed so that it is not visible from the outside. The remaining area 7 remains visible in this condition.

The line between the stowing area 6 and the remaining area 7 is an identification 8 that allows the user to distinguish the stowing area 6 from the remaining area 7. This way the user can see whether the pressing force is adequate or if he must increase or reduce the pressing force. The user can optimize the pressing force to generate a good sealing between the mask 1 and the face.

FIG. 1a shows an embodiment of the identification 8 shown in FIG. 1. Here the identification 8 is realized by a surface structure that is formed on the stowing area 6. In this case the surface structure shows the form of waves.

FIG. 1b shows an embodiment of the identification 8 shown in FIG. 1. The identification 8 is realized by a surface structure that is formed on the stowing area 6 as well. In this embodiment the surface structure shows the form of bubbles.

FIG. 1c shows an identification 8 that is realized by a difference in colors between the stowing area 6 and the remaining area 7.

FIG. 1d shows an identification 8 that is formed by a line, rib, groove or notch being arranged between the stowing area 6 and the remaining area 7.

There are more options available for allowing the user to distinguish between a stowing area 6 and a remaining area 7, like for example different roughnesses of the areas or a glossy finish. The coloring can be provided only in some areas of the identification 8, so that for example stripes or dots appear in the identification 8 area. Also, it is possible to provide different surface structures or textures. A stowing area 6 or a remaining area 7 may be transparent. Certainly, it is possible to combine several options.

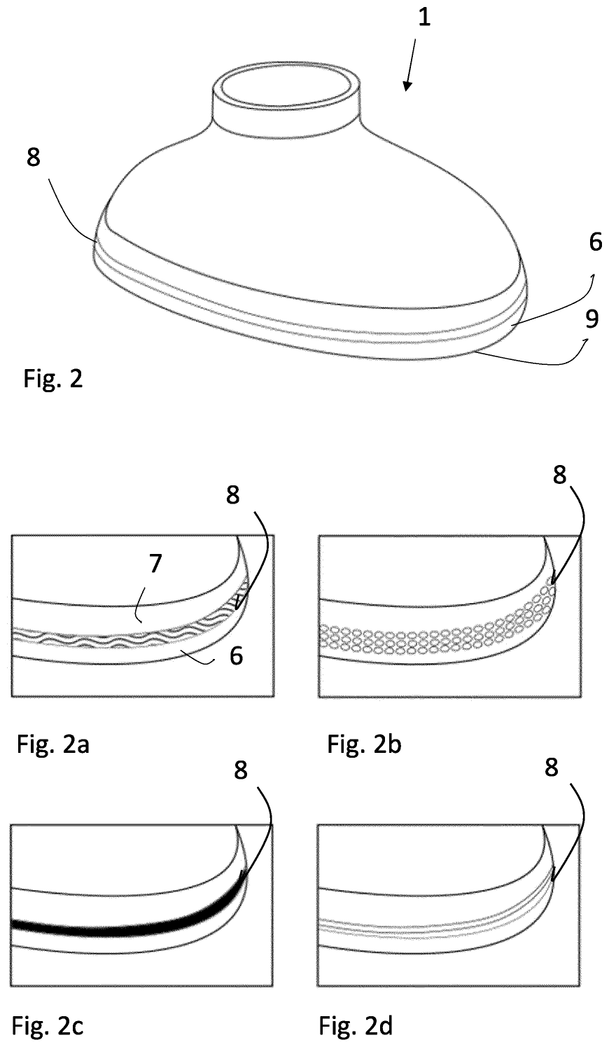

FIG. 2 shows an inhalation mask 1 like the mask shown in FIG. 1 with an identification 8 being arranged on the stowing area 6 in a distance from the sealing border 9.

When the mask 1 is pressed onto a face with an adequate force the identification 8 cannot be seen from the user anymore. It lays on the face or is compressed so that it is not visible from the outside. When the user lays the mask 1 onto the face and starts increasing pressure, at first the stowing area 6 without the identification 8 will disappear. After that with still increasing pressure the area with identification 8 will disappear. Thus, the user sees that he is close to the optimized pressure when the area with the identification 8 starts disappearing. The user knows that the pressure increase needed is only very small. Therefore, he can act more carefully from this point avoiding uncomfortable pressure for the patient.

FIG. 2a shows an embodiment of the identification 8 shown in FIG. 2. Here, the identification 8 is realized by a surface structure that is formed on the section of the stowing area 6 that is adjoining the remaining area 7. In this case the surface structure shows the form of waves.

FIG. 2b shows an embodiment of the identification 8 like the identification 8 shown in FIG. 2a. Here, the identification 8 is realized by a surface structure showing bubbles. The bubbles cover a broad area around the contact area of the stowing area and the remaining area. This way a transition area for an adequate pressure is marked. It may be easier for user to adjust the pressure according to the transition area and avoid stress by trying to apply an ideal pressure.

FIG. 2c shows an embodiment of the identification 8 like the identification 8 shown in FIGS. 2a and 2b. Here, the identification 8 is realized by a different color of the identification.

FIG. 2d shows an identification 8 arranged in the same area as the identification 8 shown in FIGS. 2a, 2b and 2c. Here the identification 8 is realized by several lines or small ribs.

It is possible to provide an identification 8 like the one shown in FIG. 2 in a slightly different area. The identification 8 may be arranged in the remaining area 7 close to the stowing area 6. This way only a section of the remaining area 7 is provided with a special structure or color.

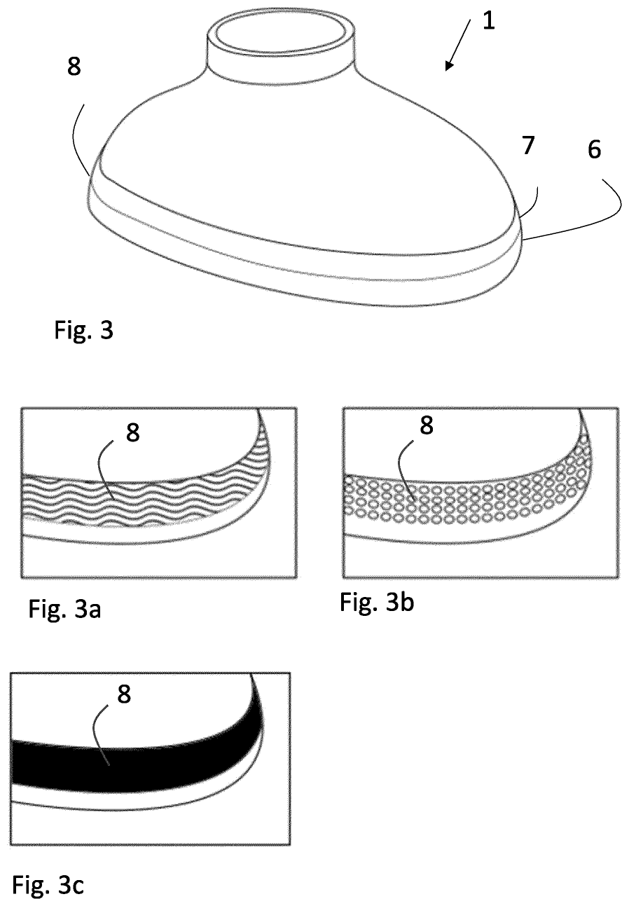

FIG. 3 shows an inhalation mask 1 like the masks shown in FIG. 1 and FIG. 2 with an identification 8 being arranged on the remaining area 7.

When the mask 1 is pressed onto a face with an adequate force the identification 8 can be seen from the user in full. The unmarked stowing area 6 lays on the face or is compressed so that it is not visible from the outside. When the user lays the mask 1 onto the face and starts increasing pressure, the stowing area 6 without the identification 8 will disappear.

FIGS. 3a, 3b and 3c show embodiments of the identification 8 like surface structures with waves or bubbles and colors.

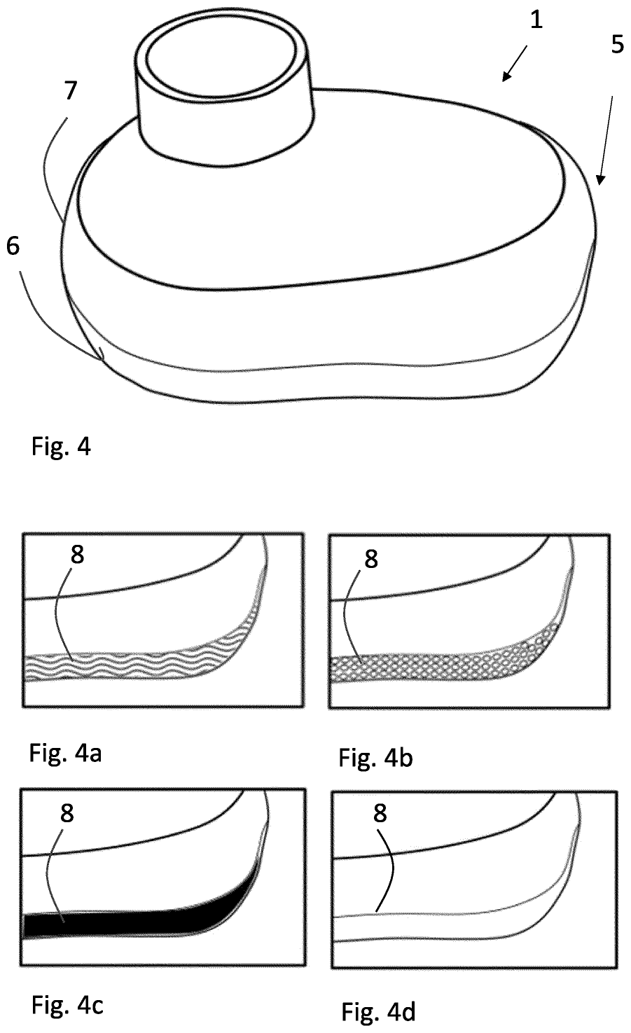

FIG. 4 shows an inhalation mask 1 like the mask shown in FIG. 1. A difference between the inhalation mask 1 shown in FIG. 1 and the inhalation mask 1 shown in FIG. 4 is the configuration of the circumferential sealing lip 5. The sealing lip 5 shown in FIG. 1 possesses a mainly flat contour and the sealing lip 5 shown in FIG. 4 shows a contour that is more adapted to a face. It provides room for a nose and the jowls. The sealing lip 5 is thicker around the chin and between the nose and the jowls and thinner around the nose and the jowls. Therefore, it provides better sealing capabilities than the mask 1 shown in FIG. 1. The stowing area 6 has the same thickness in all areas. The remaining area 7 is thinner around the nose and the jowls than in the other areas. It is possible to allocate the sealing material in other ways to the stowing area 6 and the remaining area 7. For example, the thickness of the remaining area 7 may be of the same thickness in all sections and the thickness of the stowing area 6 may be adapted to the face contour. Also, it is possible to adapt both areas to the face contour.

FIGS. 4a, 4b, 4c, and 4d show different embodiments of the identification 8 as FIGS. 1a, 1b, 1c and 1d. As shown in the description of FIGS. 1, 1a, 1b, 1c and 1d many options for marking the identification 8 are possible. The surface structures waves and bubbles, the coloring and the line or rib are just examples.

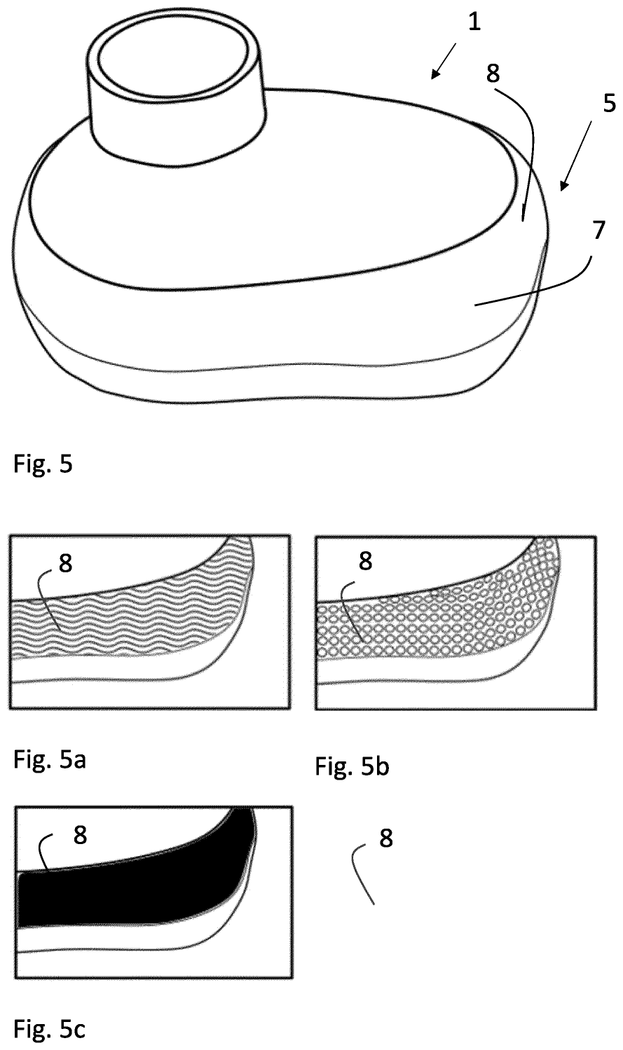

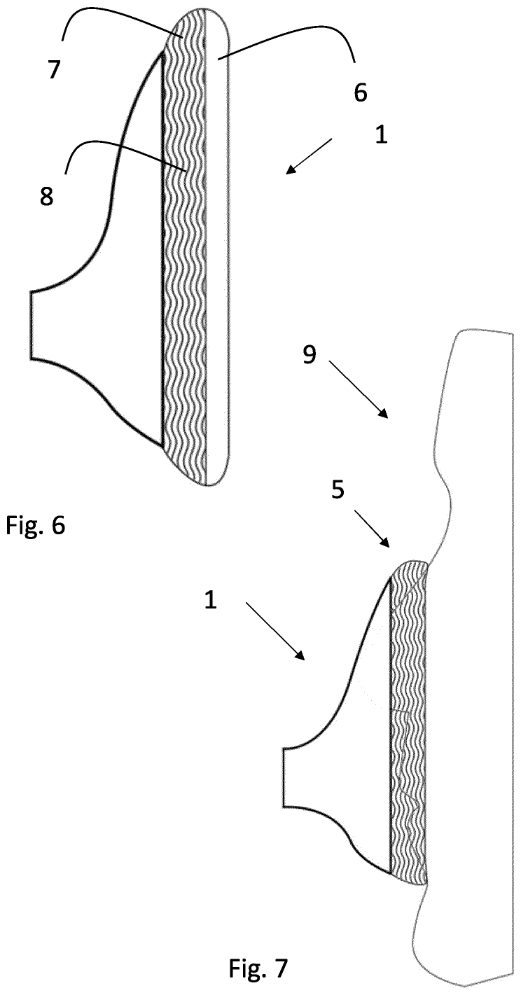

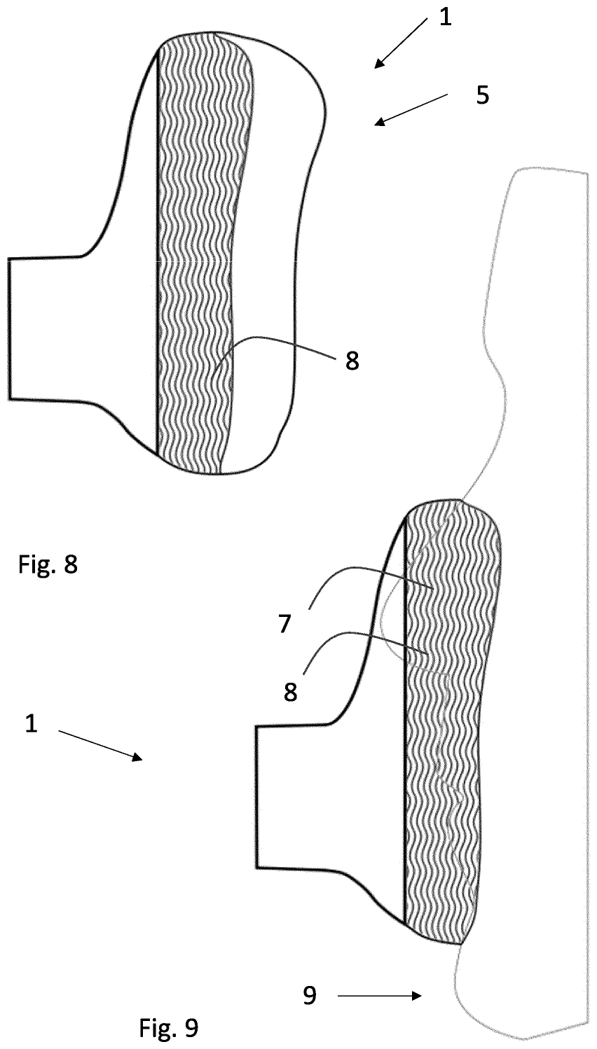

FIG. 5 shows an inhalation mask 1 like the mask shown in FIG. 3. A difference between the inhalation mask 1 shown in FIG. 3 and the inhalation mask 1 shown in FIG. 5 is the configuration of the circumferential sealing lip 5. The sealing lip 5 is formed like the sealing lip 5 shown in FIG. 4. The identification 8 is arranged on the remaining area 7 like in mask 1 shown in FIG. 3.

FIGS. 5a, 5b and 5c show different embodiments of the identification 8 that can be used with the mask 1 shown in FIG. 5. These embodiments are equivalent to the embodiments described with FIGS. 3a, 3b and 3c. These embodiments are examples. As described above many embodiments for the identification 8 are possible.

FIG. 6 shows a sideview of a mask 1 like mask 1 shown in FIG. 3 with an embodiment of the identification 8 as shown in FIG. 3a. The identification 8 marks the remaining area 7. Adjacent to the identification 8 is the stowing area 6. The identification 8 is a marking for visualization of the deformation trajectory or deformation path. It shows to what extend the inhalation mask 1 has to be deformed in order to provide a good sealing.

FIG. 7 shows the mask 1 shown in FIG. 6 being pressed on a face 9 with an optimized pressure force. The mask is deformed and the stowing area 6 is not viewable. It lays on the face 9 or is compressed. In this condition the mask 1 shows a good sealing condition and does not hurt the patient. The user pressing the mask 1 on the face 9 gets feedback regarding the pressuring force. He sees how much material of the sealing lip 5 should disappear and can adjust the pressure accordingly.

FIG. 8 shows a side view of a mask 1 like mask 1 shown in FIG. 6. The difference between the two masks 1 is that FIG. 8 shows a sealing lip 5 being adapted to the contour of the face. Masks with adapted contour are described with FIGS. 4 and 5. The embodiment of the identification 8 is described with FIG. 5a.

FIG. 9 shows the mask 1 shown in FIG. 8 being pressed onto a face 9 with an optimized pressure force. The difference to the situation shown in FIG. 7 is that the contour of the remaining area 7 is adapted to the face 9 in a better way.

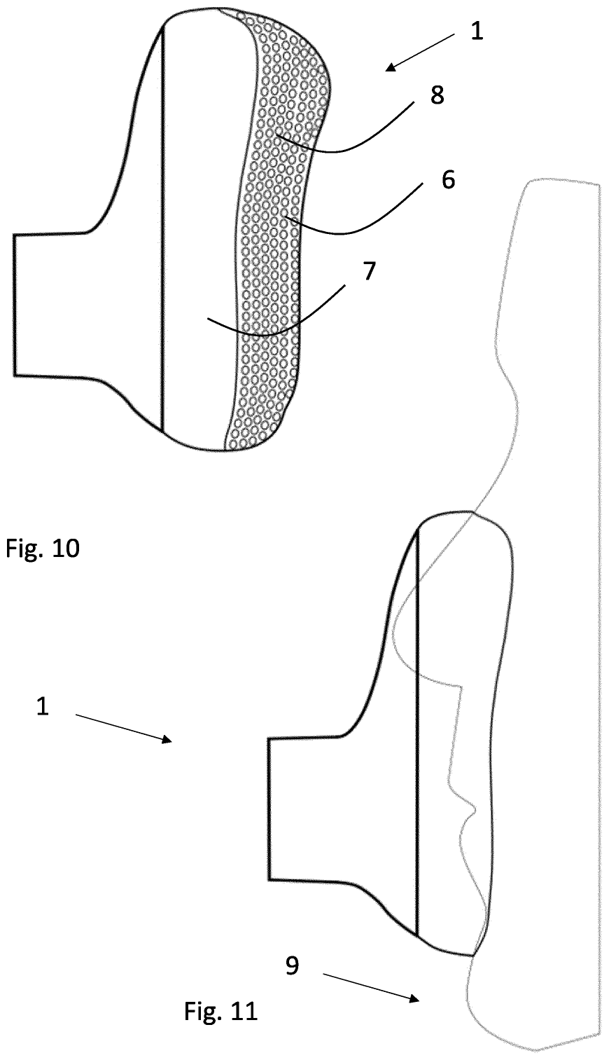

FIG. 10 shows a side view of a mask 1 like mask 1 shown in FIG. 8. A difference to the mask 1 shown in FIG. 8 is the identification 8 being arranged in the stowing area 6 instead of the remaining area 7. Another difference is that the identification 8 is realized by bubbles instead of waves.

FIG. 11 shows the mask 1 shown in FIG. 10 being pressed onto a face 9 with an optimized pressure force. The identification 8 on the stowing area 6 is not viewable from the outside anymore.

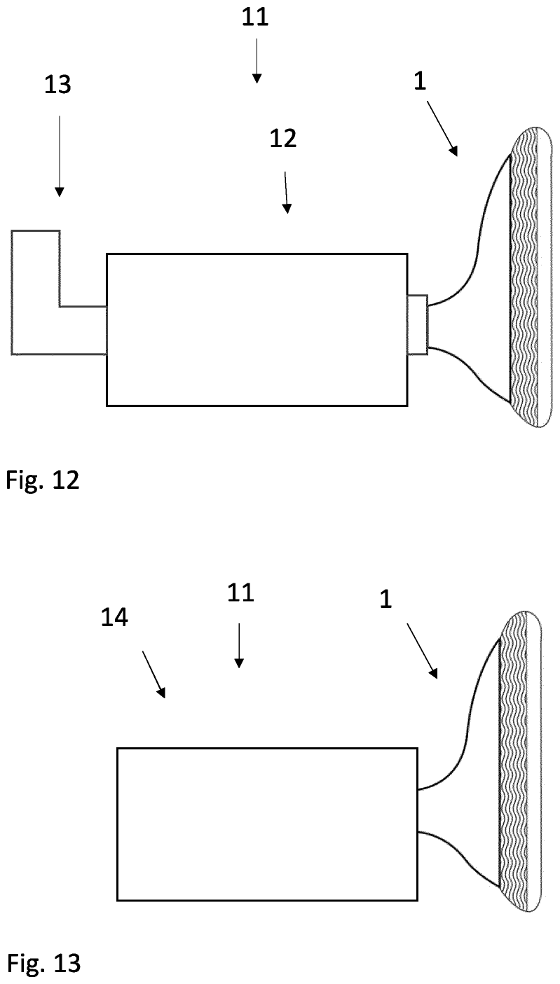

FIG. 12 shows an inhalation assembly 11 comprising a mask 1, a chamber 12 and a metered-dose inhaler (MDI) 13. Upon activation the MDI 13 releases an aerosol into the chamber 12. Chamber 12 is attached to the mask 1 which can be applied to a patient's face, so that the patient can inhale the aerosol.

FIG. 13 shows an inhalation assembly 11 comprising a mask 1 and an inhalation system 14. The inhalation system 14 may comprise a nebulizer like a jet nebulizer, a vibrating membrane nebulizer or any other inhalation system 14. Inhalation system 14 is attached to the mask 1 which can be applied to a patient's face, so that the patient can inhale a medicament, gas, aerosol or whatever the inhalation system 14 provides.

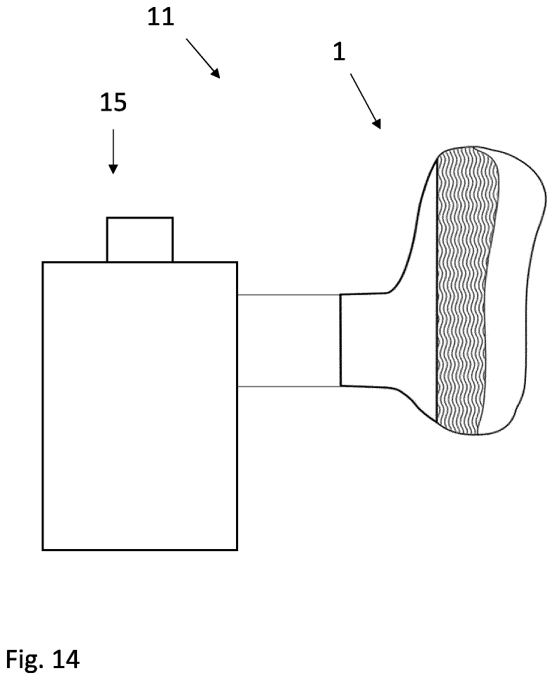

FIG. 14 shows an inhalation assembly 11 comprising a mask 1 and a nebulizer 15. In this example nebulizer 15 is a jet nebulizer.

### LIST OF REFERENCE SIGNS

1 mask
2 mask body
3 port
4 opening
5 sealing lip
6 stowing area
7 remaining area
8 identification
9 face
11 inhalation assembly
12 chamber
13 MDI
14 inhalation system
15 nebulizer

1. Inhalation mask (1) for an inhalation system (14) comprising
a mask body (2) with
a port (3) that can be connected to the inhalation system (14), and
an opening (4) with a sealing lip (5) being equipped for joining a face (9) in such a way that the nose, the mouth or both of them are surrounded by the sealing lip (5),
the sealing lip (5) comprising a stowing area (6) and a remaining area (7) arranged adjacent to the stowing area (6),
wherein
in a sealing position the stowing area (6) shows at least in one section one or more of the conditions laying on the face, being compressed or bent to an inner side of the inhalation mask (1) and
the inhalation mask (1) is provided with an identification (8) distinguishing the stowing area (6) from the remaining area (7).

2. Inhalation mask (1) according to claim 1, characterized in that the identification (8) comprises two different materials.

3. Inhalation mask (1) according to claim 1, characterized in that the identification (8) comprises a first color for the stowing area (6) and a different color for the remaining area (7).

4. Inhalation mask (1) according to claim 1, characterized in that the identification (8) comprises a marking or a surface structure.

5. Inhalation mask (1) according to claim 4, characterized in that the marking or surface structure comprises several elements.

6. Inhalation mask (1) according to claim 5, characterized in that the marking or surface structure is at least to some extend provided in the remaining area (7), characterized in that the dimensions of the elements are related to their distance to the stowing area (6).

7. Inhalation mask (1) according to claim 4, characterized in that the surface structure comprises a rib, notch or groove.

8. Inhalation mask (1) according to claim 1, characterized in that the identification (8) is invisible from the outside of the mask body (2) when the mask (1) is pressed on the face (9) in a correct way.

9. Inhalation assembly (11) comprising an inhalation mask (1) according to claim 1 and an inhalation system (14).

10. Method for using an inhalation mask (1) according to claim,
comprising the steps of
pressing the inhalation mask (1) on a face (9) in such a way that the nose, the mouth or both of them are surrounded by the sealing lip (5) and
adjusting the pressure by watching the identification (8) such that the stowing area (6) is deformed such that it is invisible from an outside of the inhalation mask (1) or loses contact to an ambient environment outside the mask and the remaining area (7) is still viewable or in contact with the ambient environment.
